(19) United States
(12) Patent Application Publication
(10) Pub. No.: US 2019/0069769 A1
(43) Pub. Date: Mar. 7, 2019

(54) LIVING BODY OBSERVATION SYSTEM

(71) Applicant: OLYMPUS CORPORATION, Tokyo (JP)

(72) Inventor: Kei KUBO, Tokyo (JP)

(73) Assignee: OLYMPUS CORPORATION, Tokyo (JP)

(21) Appl. No.: 16/175,923

(22) Filed: Oct. 31, 2018

Related U.S. Application Data
(63) Continuation of application No. PCT/JP2017/006766, filed on Feb. 23, 2017.

(30) Foreign Application Priority Data
May 19, 2016 (JP) 2016-100594

(51) Int. Cl. A61B 1/06; A61B 1/045; A61B 1/00

(52) U.S. Cl. CPC A61B 1/0676; A61B 5/02042; A61B 1/00096; A61B 1/045

(57) ABSTRACT

A living body observation system includes a light source apparatus, a camera unit having a plurality of pixels that receive light from a subject to generate an image pickup signal, an image processing section and a control section. The image processing section has a white light observation image generation mode and a deep blood vessel observation image generation mode, and generates color images of the respective modes. The control section performs control to switch an observation image generation mode to the deep blood vessel observation image generation mode from the white light observation image generation mode when a size of a region of blood in a color image becomes equal to or more than a predetermined value.

### CROSS REFERENCE TO RELATED APPLICATION

This application is a continuation application of PCT/JP2017/006766 filed on Feb. 23, 2017 and claims benefit of Japanese Application No. 2016-100594 filed in Japan on May 19, 2016, the entire contents of which are incorporated herein by this reference.

### BACKGROUND OF THE INVENTION

### 1. Field of the Invention

The present invention relates to a living body observation system, and particularly relates to a living body observation system capable of switching an observation mode.

### 2. Description of Related Art

Endoscope apparatuses that obtain endoscope images of insides of body cavities by emitting illuminating light are widely used. A surgeon can perform various diagnoses and necessary treatments while viewing an endoscope image of a biological tissue displayed on a monitor by using an endoscope apparatus.

Some endoscope apparatuses used as living body observation systems have a plurality of observation modes such as a normal light observation mode for observing a biological tissue by illuminating the biological tissue with white light as illuminating light, and a special light observation mode of observing a biological tissue by illuminating the biological tissue with special light as illuminating light.

Japanese Patent Application Laid-Open Publication No. 2006-341078 proposes an endoscope apparatus that makes it possible to set generation characteristics and the like of a spectral image to obtain observation images of suitable color tones even when the kinds of mucosal tissues to be observed are different.

Further, Japanese Patent Application Laid-Open Publication No. 2012-152333 proposes an endoscope system that controls a light source that emits illuminating light so that an endoscope image suitable for observation can be obtained in accordance with the kind of a biological tissue which is an object to be observed.

Incidentally, when a surgeon perfoinis treatment to a biological tissue while viewing an endoscope image, the surgeon performs treatment so as not to hurt a blood vessel and the like, but bleeding may occur. When bleeding occurs, the surgeon finds out a bleeding point, and performs a hemostasis treatment on the bleeding point by using a high-frequency scalpel or a hemostatic forceps.

Under white light, the whole blood is displayed in a red color tone, so that it is difficult to recognize the bleeding point visually, but by switching an observation mode to an observation mode that enables observation of a blood vessel in a mucosal deep part by using light in a red band, the surgeon can visually recognize the bleeding point.

### SUMMARY OF THE INVENTION

A living body observation system of one aspect of the present invention includes a light source section configured to be able to switch between a first illumination mode that emits light which is illuminating light for irradiating a subject to generate a first observation image, as the illuminating light, and a second illuminating mode that emits a light in a first wavelength band that is a red band in a visible region and is a narrow band between a wavelength band including a maximum value and a wavelength band including a minimum value in a hemoglobin light absorption characteristic of a biological tissue of the subject, a light in a second wavelength band that is a wavelength band having a longer wavelength than the first wavelength band and is a narrow band in which an absorption coefficient in the hemoglobin light absorption characteristic is lower than the light in the first wavelength band and a scattering characteristic of the biological tissue is suppressed, and a light in a third wavelength band of a shorter wavelength than the red band in the visible region, as the illuminating light, an image pickup section including a plurality of pixels that receive light from the subject and generate an image pickup signal, a color image generation section configured to include a first observation image generation mode that generates the first observation image of the subject from the image pickup signal, and a second observation image generation mode that generates a second observation image that is a deep blood vessel observation image that is for observing a deep blood vessel of the subject and is obtained when the light in the first wavelength band, the light in the second wavelength band, and the light in the third wavelength band are irradiated, as observation image generation modes, and generate color images of the subject in the first observation image generation mode and the second observation image generation mode respectively, and a control section configured to calculate a size of a bleeding region from a bleeding point based on a pixel value of an image pickup signal corresponding to the light in the first wavelength band included in the image pickup signal generated in the image pickup section in the second observation image generation mode, and perform control to switch the observation image generation mode in the color image generation section to the first observation image generation mode from the second observation image generation mode when the size of the bleeding region from the bleeding point becomes equal to or less than a first value, and switch the observation image generation mode in the color image generation section to the second observation image generation mode from the first observation image generation mode when the size of the bleeding region in the color image that is generated by the color image generation section becomes equal to or more than a second value.

### BRIEF DESCRIPTION OF THE DRAWINGS

FIG. 1 is a view illustrating a configuration of a main part of a living body observation system according to an embodiment of the present invention;

FIG. 2 is a diagram for explaining one example of a specific configuration of the living body observation system according to the embodiment of the present invention;

FIG. 3 is a diagram illustrating an example of an optical characteristic of a dichroic mirror provided in a camera unit of an endoscope, according to the embodiment of the present invention;

FIG. 4 is a diagram illustrating an example of a sensitivity characteristic of an image pickup device provided in the camera unit of the endoscope according to the embodiment of the present invention;

FIG. 5 is a diagram illustrating an example of the sensitivity characteristic of the image pickup device provided in the camera unit of the endoscope, according to the embodiment of the present invention;

FIG. 6 is a diagram illustrating examples of lights that are emitted from respective light sources provided in a light source apparatus, according to the embodiment of the present invention;

FIG. 7 is a diagram for explaining an example of a specific configuration of an image processing section provided in a processor, according to the embodiment of the present invention;

FIG. 8 is a flowchart illustrating an example of a flow of a switching process of an observation mode by a control section 44, according to the embodiment of the present invention;

FIG. 9 is a diagram for explaining a bleeding region in an observation image OG (N) in a white light observation mode, according to the embodiment of the present invention;

FIG. 10 is a diagram for explaining a bleeding region and a bleeding point in an observation image OG (D) in a deep blood vessel mode, according to the embodiment of the present invention;

FIG. 11 is a schematic graph illustrating a light absorption characteristic of blood to a light wavelength, according to the embodiment of the present invention; and

FIG. 12 is a diagram for explaining a change of an observation image by switching of an observation mode, according to the embodiment of the present invention.

### DETAILED DESCRIPTION OF PREFERRED EMBODIMENT

An embodiment of the present invention will be described below with reference to the drawings.

### (Configuration)

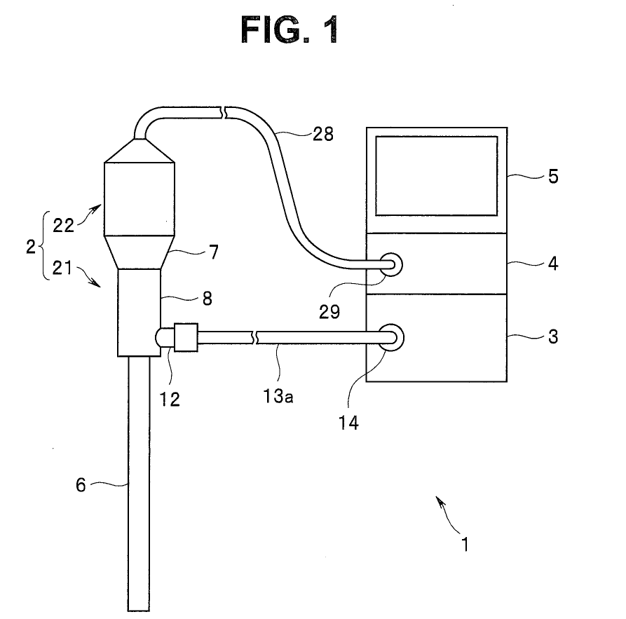

As illustrated in FIG. 1, a living body observation system 1 that is an endoscope apparatus has an endoscope 2 configured to be inserted into a subject and pick up an image of an object such as a biological tissue in the subject to output an image signal, a light source apparatus 3 configured to supply light that is emitted to the object to the endoscope 2, a processor 4 configured to generate an observation image based on the image signal which is outputted from the endoscope 2 to output the observation image, and a display apparatus 5 configured to display the observation image that is outputted from the processor 4 on a screen. FIG. 1 is a view illustrating a configuration of a main part of the living body observation system according to the embodiment. In this case, the living body observation system 1 has two observation modes that are a white light observation image generation mode and a deep blood vessel observation image generation mode, as observation image generation modes.

The endoscope 2 is configured by having a telescope 21 including an elongated insertion portion 6, and a camera unit 22 that is attachable to and detachable from an eyepiece portion 7 of the telescope 21.

The telescope 21 is configured by having the elongated insertion portion 6 insertable into a subject, a grasping portion 8 provided at a proximal end portion of the insertion portion 6, and the eyepiece portion 7 provided at a proximal end portion of the grasping portion 8.

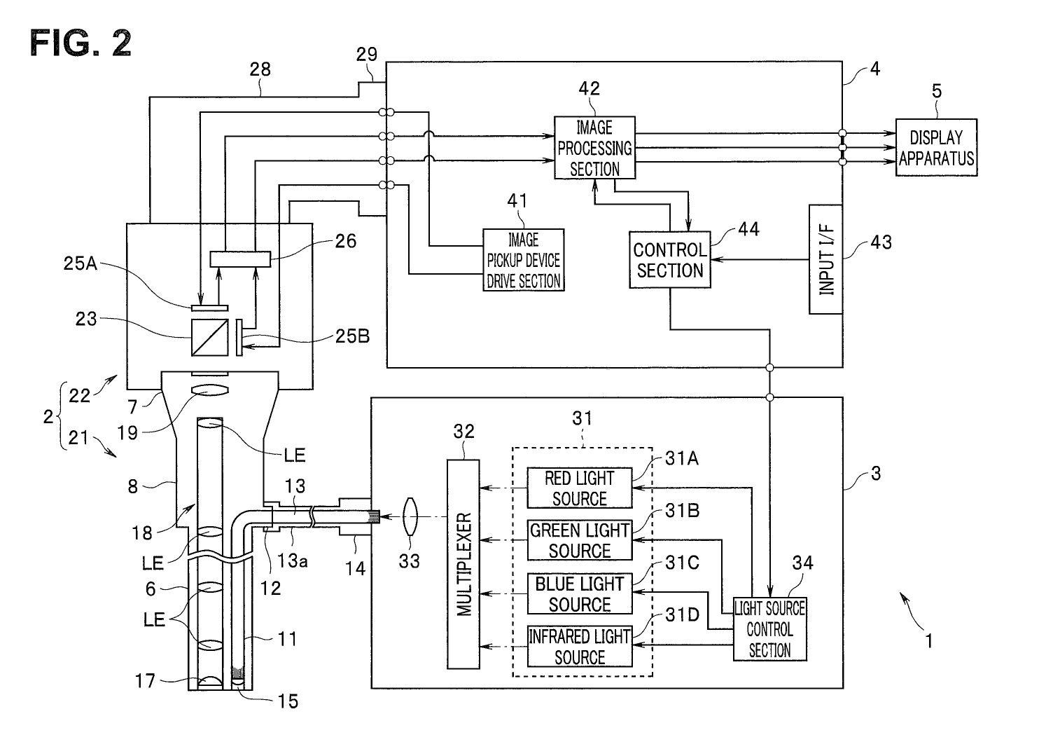

As illustrated in FIG. 2, a light guide 11 for transmitting light that is supplied via a cable 13a is inserted through an inside of the insertion portion 6. FIG. 2 is a diagram for explaining an example of a specific configuration of the living body observation system according to the embodiment.

An emission end portion of the light guide 11 is disposed in a vicinity of an illumination lens 15 in a distal end portion of the insertion portion 6 as illustrated in FIG. 2. Further, an incidence end portion of the light guide 11 is disposed in a light guide base 12 provided in the grasping portion 8.

As illustrated in FIG. 2, a light guide 13 for transmitting light that is supplied from the light source apparatus 3 is inserted through an inside of the cable 13a. Further, a connection member (not illustrated) that is attachable to and detachable from the light guide base 12 is provided at one end portion of the cable 13a. Further, a light guide connector 14 that is attachable to and detachable from the light source apparatus 3 is provided at the other end portion of the cable 13a.

The illumination lens 15 for emitting the light transmitted by the light guide 11 to outside, and an objective lens 17 for obtaining an optical image corresponding to light that is incident from outside are provided at the distal end portion of the insertion portion 6. Further, on a distal end surface of the insertion portion 6, an illumination window (not illustrated) in which the illumination lens 15 is disposed, and an observation window (not illustrated) in which the objective lens 17 is disposed are provided adjacently to each other.

As illustrated in FIG. 2, a relay lens 18 including a plurality of lenses LE for transmitting the optical image obtained by the objective lens 17 to the eyepiece portion 7 is provided inside the insertion portion 6. That is, the relay lens 18 is configured by including a function as a transmission optical system that transmits light incident from the objective lens 17.

As illustrated in FIG. 2, an eyepiece lens 19 for enabling the optical image transmitted by the relay lens 18 to be observed with the naked eye is provided inside the eyepiece portion 7.

The camera unit 22 is configured by having a dichroic mirror 23, and image pickup devices 25A and 25B.

The dichroic mirror 23 is configured to transmit light in a visible region included in the emission light that is emitted through the eyepiece lens 19 to an image pickup device 25A side, and reflect light in a near-infrared region included in the emission light to an image pickup device 25B side.

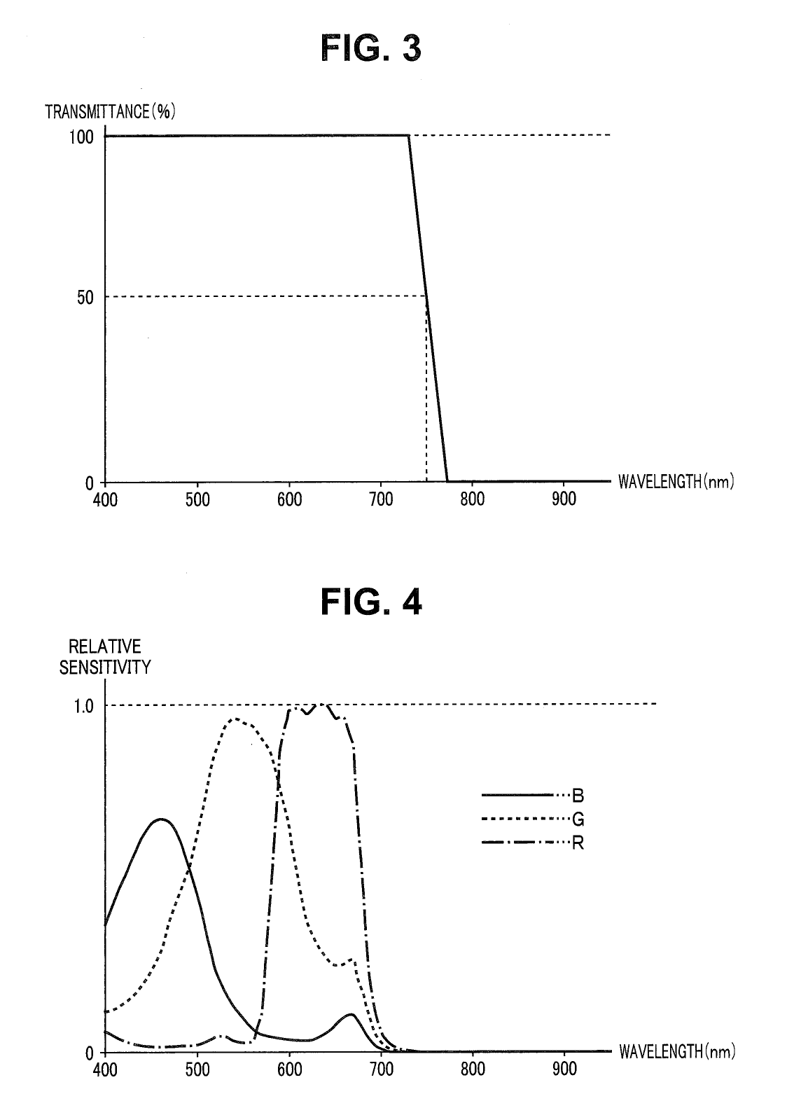

The dichroic mirror 23 is configured so that a spectral transmittance in a wavelength band belonging to a visible region is 100% as illustrated in FIG. 3, for example. Further, the dichroic mirror 23 is configured so that a half value wavelength that is a wavelength at which the spectral transmittance=50% is established is 750 nm, as illustrated in FIG. 3, for example. FIG. 3 is a diagram illustrating an example of an optical characteristic of the dichroic mirror provided in the camera unit of the endoscope according to the embodiment.

That is, the dichroic mirror 23 is configured to have a function as a spectral optical system, and emit light that is emitted through the eyepiece lens 19 by separating the light into lights of two wavelength bands that is a light in a visible region and a light in a near-infrared region.

Note that the dichroic mirror 23 may be configured so that the half value wavelength is another wavelength different from 750 nm as long as the dichroic mirror 23 has the function of the aforementioned spectral optical system.

The image pickup device 25A is configured by having a color CCD, for example. Further, the image pickup device 25A is disposed in a position where the image pickup device 25A can receive the light in a visible region which is transmitted through the dichroic mirror 23, inside the camera unit 22. Further, the image pickup device 25A is configured by including a plurality of pixels for photoelectrically converting the light in the visible region transmitted through the dichroic mirror 23 and picking up an image of the light, and a primary color filter provided on an image pickup surface on which the plurality of pixels are disposed two-dimensionally. Further, the image pickup device 25A is configured to be driven in response to an image pickup device drive signal that is outputted from the processor 4, generate an image pickup signal by picking up the light in the visible region that is transmitted through the dichroic mirror 23, and output the generated image pickup signal to a signal processing circuit 26.

The image pickup device 25A is configured by including a sensitivity characteristic as illustrated in FIG. 4 in respective wavelength bands of R (red), G (green) and B (blue). That is, the image pickup device 25A is configured not to have or hardly have sensitivity in wavelength bands outside the visible region while having sensitivity in the visible region including the respective wavelength bands of R, G and B. FIG. 4 is a diagram illustrating an example of the sensitivity characteristic of the image pickup device provided in the camera unit of the endoscope according to the embodiment.

The image pickup device 25B is configured by including a monochrome CCD, for example. Further, the image pickup device 25B is disposed in a position where the image pickup device 25B can receive the light in the near-infrared region that is reflected by the dichroic mirror 23, inside the camera unit 22. Further, the image pickup device 25B is configured by including a plurality of pixels for photoelectrically converting the light in the near-infrared region that is reflected by the dichroic mirror 23 and picking up an image. Further, the image pickup device 25B is configured to be driven in response to an image pickup device drive signal that is outputted from the processor 4, generate an image pickup signal by picking up an image of the light in the near-infrared region that is reflected by the dichroic mirror 23, and output the generated image pickup signal to the signal processing circuit 26.

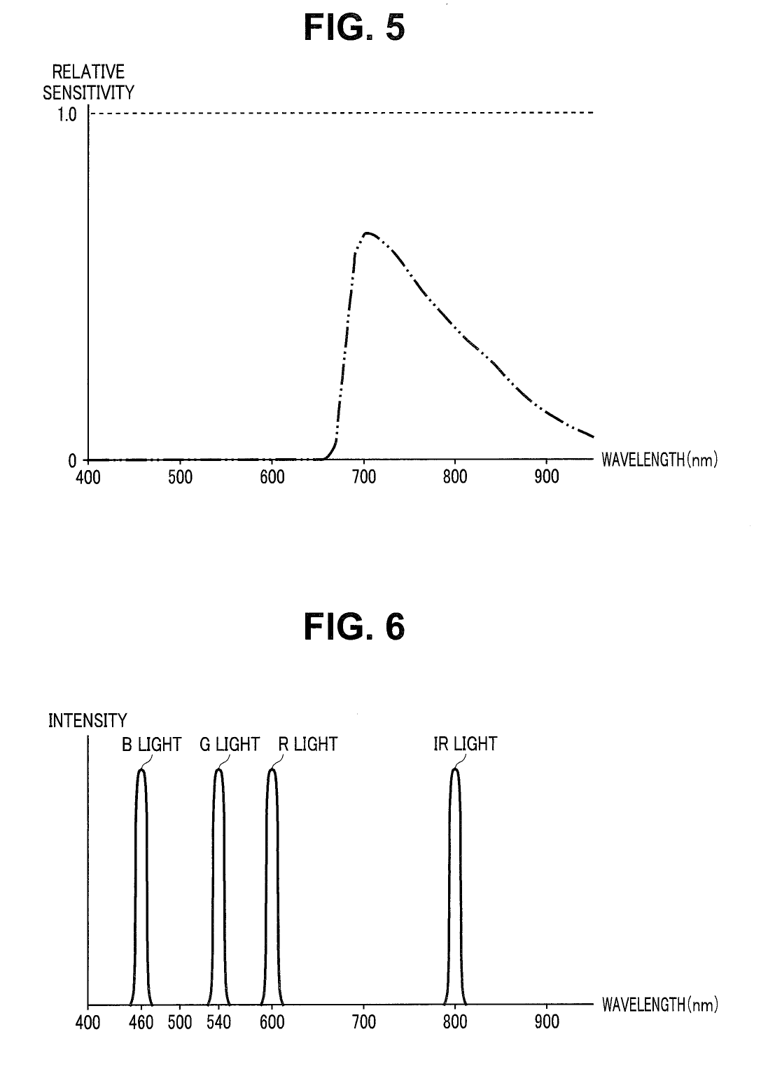

The image pickup device 25B is configured by including a sensitivity characteristic as illustrated in FIG. 5 in the near-infrared region. More specifically, the image pickup device 25B is configured to have sensitivity in the near-infrared region including at least 700 nm to 900 nm while the image pickup device 25B does not have or hardly has sensitivity in the visible region including the respective wavelength bands of R, G and B, for example. FIG. 5 is a diagram illustrating an example of the sensitivity characteristic of the image pickup device provided in the camera unit of the endoscope according to the embodiment.

Consequently, the image pickup devices 25A and 25B configure an image pickup section having a plurality of pixels that receive light from the subject irradiated with the illuminating light to generate an image pickup signal.

The signal processing circuit 26 is configured to generate an image signal CS including at least one of an image of a red component (also referred to as an R image hereinafter), an image of a green component (also referred to as a G image hereinafter) and an image of a blue component (also referred to as a B image hereinafter) by applying predeteimined signal processes such as a correlated double sampling process, and an A/D conversion process to the image pickup signal that is outputted from the image pickup device 25A, and output the generated image signal CS to the processor 4 to which a signal cable 28 is connected. A connector 29 is provided at an end portion of the signal cable 28, and the signal cable 28 is connected to the processor 4 via the connector 29. The signal processing circuit 26 is configured to generate an image signal IRS corresponding to an image of a near-infrared component (also referred to as an IR image hereinafter) by applying the predeteithined signal processes such as a correlated double sampling process and an A/D conversion process to the image pickup signal which is outputted from the image pickup device 25B, and output the generated image signal IRS to the processor 4 to which the signal cable 28 is connected.

Note that in the following explanation, explanation will be advanced by citing a case where the R image and the B image that are included in the image signal CS have a same resolution RA, and the IR image shown by the image signal IRS has a larger resolution RB than the resolution RA as an example, for simplification.

The light source apparatus 3 is a light source section that generates illuminating light for illuminating a subject, and is configured by having a light emission section 31, a multiplexer 32, a condensing lens 33, a light source control section 34.

The light emission section 31 is configured by having a red light source 31A, a green light source 31B, a blue light source 31C and an infrared light source 31D.

The red light source 31A is configured by including a lamp, an LED or an LD, for example. Further, the red light source 31A is configured to emit R light that is a narrow band light that belongs to a red band in a visible region, and has a center wavelength and a bandwidth respectively set between a wavelength band including a maximum value and a wavelength band including a minimum value in a light absorption characteristic of hemoglobin of the biological tissue of the subject. More specifically, as illustrated in FIG. 6, the red light source 31A is configured to emit R light in which the center wavelength is set at a vicinity of 600 nm and a bandwidth is set at 20 nm. FIG. 6 is a diagram illustrating examples of lights that are emitted from the respective light sources provided in the light source apparatus according to the embodiment.

Note that the center wavelength of the R light does not need to be set at the vicinity of 600 nm, and can be set at a wavelength WR that belongs to a range between 580 and 620 nm, for example. Further, the bandwidth of the R light does not need to be set at 20 nm, and can be set at a predeteimined bandwidth corresponding to the wavelength WR, for example.

The red light source 31A is configured to switch to a lighting state or an extinguishing state in accordance with control of the light source control section 34. Further, the red light source 31A is configured to generate the R light with an intensity corresponding to the control of the light source control section 34 in the lighting state.

The green light source 31B is configured by including a lamp, an LED or an LD, for example. Further, the green light source 31B is configured to emit G light that is a narrow band light that belongs to a green region. More specifically, the green light source 31B is configured to emit the G light in which the center wavelength is set at a vicinity of 540 nm, and a bandwidth is set at 20 nm as illustrated in FIG. 6.

Note that the center wavelength of the G light can be set at a wavelength WG that belongs to a green region. Further, the bandwidth of the G light does not need to be set at 20 nm, and can be set at a predeteiinined bandwidth corresponding to the wavelength WG, for example.

The green light source 31B is configured to switch to a lighting state or an extinguishing state in accordance with control of the light source control section 34. Further, the green light source 31B is configured to generate the G light with an intensity corresponding to control of the light source control section 34 in the lighting state.

The blue light source 31C is configured by including a lamp, an LED or an LD, for example. Further, the blue light source 31C is configured to emit B light that is a narrow band light that belongs to a blue region. More specifically, the blue light source 31C is configured to emit a light with a shorter wavelength than the red band in the visible region, and emit the B light in which a center wavelength is set at a vicinity of 460 nm and a bandwidth is set at 20 nm, as illustrated in FIG. 6.

Note that the center wavelength of the B light may be set at a vicinity of 470 nm, for example, as long as the center wavelength is set at a wavelength WB that belongs to the blue region. Further, the bandwidth of the B light does not need to be set at 20 nm, and can be set at a predetermined bandwidth corresponding to the wavelength WB, for example.

The blue light source 31C is configured to switch to a lighting state or an extinguishing state in accordance with control of the light source control section 34. Further, the blue light source 31C is configured to generate the B light with an intensity corresponding to control of the light source control section 34 in the lighting state.

The infrared light source 31D is configured by including a lamp, an LED or an LD, for example. Further, the infrared light source 31D is configured to emit IR light that is a narrow band light that belongs to a near-infrared region, and has a center wavelength and a bandwidth respectively set so that an absorption coefficient in a light absorption characteristic of hemoglobin is lower than an absorption coefficient of the wavelength WR (600 nm, for example), and a scattering characteristic of the biological tissue is suppressed. That is, the IR light is a narrow band light that is in a wavelength band of a longer wavelength than the R light, and has the absorption coefficient in the hemoglobin light absorption characteristic lower than the R light, and the scattering characteristic of the biological tissue suppressed. More specifically, as illustrated in FIG. 6, the infrared light source 31D is configured to emit the IR light in which the center wavelength is set at a vicinity of 800 nm and a bandwidth is set at 20 nm.

Note that the aforementioned expression “scattering characteristic of the biological tissue is suppressed” is assumed to include a meaning that “the scattering coefficient of the biological tissue becomes lower toward the long wavelength side”. Note that the center wavelength of the IR light does not need to be set at the vicinity of 800 nm, and can be set at a wavelength WIR that belongs to a wavelength between 790 to 810 nm, for example. Further, the bandwidth of the IR light does not need to be set at 20 nm, and can be set at a predetermined bandwidth corresponding to a wavelength WIR, for example.

The infrared light source 31D is configured to switch to a lighting state or an extinguishing state in accordance with control of the light source control section 34. Further, the infrared light source 31D is configured to generate the IR light with an intensity corresponding to control of the light source control section 34 in the lighting state.

The multiplexer 32 is configured to multiplex the respective lights emitted from the light emission section 31 to enable the lights to be incident on the condensing lens 33.

The condensing lens 33 is configured to condense light incident through the multiplexer 32 to emit the light to the light guide 13.

The light source control section 34 is configured to perform control to the respective light sources of the light emission section 31 based on a system control signal that is outputted from the processor 4.

The light source apparatus 3 has two illumination modes that are an illumination mode for a white light observation image generation mode (referred to as a white light mode hereinafter) and an illumination mode for a deep blood vessel observation image generation mode (referred to as a deep blood vessel mode hereinafter), and it is possible to switch between the two illumination modes.

The white light mode is a mode in which a white light observation image, which is obtained when a subject is irradiated with a white light as the illuminating light, is generated and is displayed on the display apparatus 5. In the light source apparatus 3, the red light source 31A, the green light source 31B and the blue light source 31C light up at a time of the illumination mode for the white light mode. The deep blood vessel mode is a mode in which a deep blood vessel observation image for observing a deep blood vessel of a subject which is obtained when the R light, the IR light and the B light are emitted, is generated and is displayed on the display apparatus 5. In the light source apparatus 3, the red light source 31A, the blue light source 31C and the infrared light source 31D light up at the time of the illumination mode for the deep blood vessel mode.

The processor 4 is configured by having an image pickup device drive section 41, an image processing section 42, an input I/F (interface) 43 and a control section 44.

The image pickup device drive section 41 is configured by including a driver circuit, for example. Further, the image pickup device drive section 41 is configured to generate image pickup device drive signals for driving the image pickup devices 25A and 25B and outputs the image pickup device drive signals.

Note that the image pickup device drive section 41 may drive the image pickup devices 25A and 25B respectively in accordance with drive command signals from the control section 44. That is, the image pickup device drive section 41 may drive the image pickup devices 25A and 25B respectively so as to drive only the image pickup device 25A at the time of the white light mode, and drive the image pickup devices 25A and 25B at the time of the deep blood vessel mode.

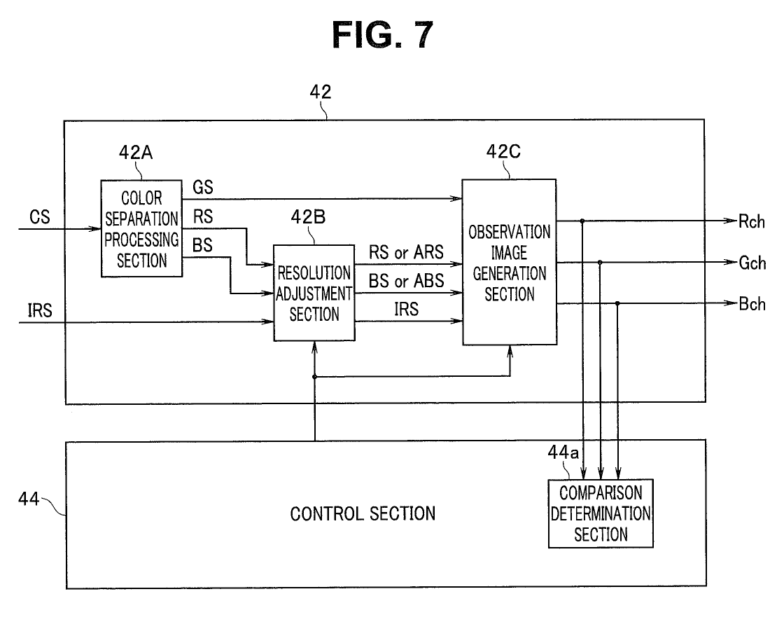

The image processing section 42 is configured by including an image processing circuit, for example. Further, the image processing section 42 is configured to generate an observation image corresponding to an observation image generation mode of the living body observation system 1 and output the observation image to the display apparatus 5, based on the image signals CS and IRS which are outputted from the endoscope 2, and a system control signal that is outputted from the control section 44. Further, the image processing section 42 is configured by having a color separation processing section 42A, a resolution adjustment section 42B, and an observation image generation section 42C as illustrated in FIG. 7, for example. FIG. 7 is a diagram for explaining an example of a specific configuration of the image processing section provided in the processor according to the embodiment.

The color separation processing section 42A is configured to perform a color separation process for separating the image signal CS which is outputted from the endoscope 2 into an R image, a G image and a B image, for example. Further, the color separation processing section 42A is configured to generate an image signal RS corresponding to the R image obtained by the aforementioned color separation process, and output the generated image signal RS to the resolution adjustment section 42B. Further, the color separation processing section 42A is configured to generate an image signal BS corresponding to the B image obtained by the aforementioned color separation process and output the generated image signal BS to the resolution adjustment section 42B. Further, the color separation processing section 42A is configured to generate the image signal GS corresponding to the G image obtained by the aforementioned color separation processing and output the generated image signal GS to the observation image generation section 42C.

The resolution adjustment section 42B is configured to output the image signals RS and BS that are outputted from the color separation processing section 42A directly to the observation image generation section 42C when the observation image generation mode is set at the white light mode, for example, based on the system control signal which is outputted from the control section 44.

The resolution adjustment section 42B is configured to perform a pixel interpolation process for increasing a resolution RA of the R image shown by the image signal RS which is outputted from the color separation processing section 42A until the resolution RA of the R image corresponds to a resolution RB of the IR image shown by the image signal IRS which is outputted from the endoscope 2, when the observation image generation mode is set at the deep blood vessel mode, for example, based on the system control signal which is outputted from the control section 44. Further, the resolution adjustment section 42B is configured to perform a pixel interpolation process for increasing the resolution RA of the B image shown by the image signal BS which is outputted from the color separation processing section 42A until the resolution RA of the B image corresponds to the resolution RB of the IR image which is shown by the image signal IRS which is outputted from the endoscope 2, when the observation image generation mode is set at the deep blood vessel mode, for example, based on the system control signal which is outputted from the control section 44.

The resolution adjustment section 42B is configured to output the image signal IRS which is outputted from the endoscope 2 directly to the observation image generation section 42C when the observation image generation mode is set at the deep blood vessel mode, for example, based on the system control signal which is outputted from the control section 44. Further, the resolution adjustment section 42B is configured to generate an image signal ARS corresponding to the R image to which the aforementioned pixel interpolation process is applied, and output the generated image signal ARS to the observation image generation section 42C, when the observation image generation mode is set at the deep blood vessel mode, for example, based on the system control signal which is outputted from the control section 44. Further, the resolution adjustment section 42B is configured to generate an image signal ABS corresponding to the B image to which the aforementioned pixel interpolation process is applied and output the generated image signal ABS to the observation image generation section 42C, when the observation image generation mode is set at the deep blood vessel mode, for example, based on the system control signal which is outputted from the control section 44.

That is, the resolution adjustment section 42B is configured to perform a process for causing the resolution of the R image shown by the image signal RS which is outputted from the color separation processing section 42A, the resolution of the B image shown by the image signal BS which is outputted from the color separation processing section 42A, and the resolution of the IR image shown by the image signal IRS which is outputted from the endoscope 2 to correspond to one another, before generation of an observation image by the observation image generation section 42C is performed, when the observation image generation mode is set at the deep blood vessel mode.

The observation image generation section 42C is configured to generate an observation image by assigning the R image shown by the image signal RS which is outputted from the resolution adjustment section 42B to an R channel corresponding to red color of the display apparatus 5, assigning the G image shown by the image signal GS which is outputted from the color separation processing section 42A to a G channel corresponding to green color of the display apparatus 5, and assigning the B image shown by the image signal BS which is outputted from the resolution adjustment section 42B to a B channel corresponding to blue color of the display apparatus 5, and output the generated observation image to the display apparatus 5, when the observation image generation mode is set at the white light mode, for example, based on the system control signal which is outputted from the control section 44.

The observation image generation section 42C is configured to generate an observation image by assigning the IR image shown by the image signal IRS which is outputted from the resolution adjustment section 42B to the R channel corresponding to the red of the display apparatus 5, assigning the R image shown by the image signal ARS which is outputted from the resolution adjustment section 42B to the G channel corresponding to the green of the display apparatus 5, and assigning the B image shown by the image signal ABS which is outputted from the resolution adjustment section 42B to the B channel corresponding to the blue of the display apparatus 5, and output the generated observation image to the display apparatus 5, when the observation image generation mode is set at the deep blood vessel mode, for example, based on the system control signal which is outputted from the control section 44.

As above, the image processing section 42 configures a color image generation section that has the white light mode that generates a white light observation image of a subject from the image pickup signal and the deep blood vessel mode that generates a deep blood vessel observation image of the subject which is different from the white light observation image from the image pickup signal as the observation image generation modes, and generates color images of the subject in the respective white light mode and deep blood vessel mode.

The input I/F 43 is configured by including one or more switches and/or buttons that can perform an instruction or the like corresponding to an operation of the surgeon who is a user. More specifically, the input I/F 43 is configured by including an observation image generation mode switching switch (not illustrated) that can perform an instruction to set (switch) the observation image generation mode of the living body observation system 1 at either one of the white light mode or the deep blood vessel mode in accordance with the operation of the user, for example.

The control section 44 is configured by including a control circuit such as a CPU or an FPGA (field programmable gate array). Further, the control section 44 is configured to generate a system control signal to cause an operation corresponding to the observation image generation mode of the living body observation system 1 to be performed based on an instruction that is made in the observation image generation mode switching switch of the input I/F 43, and output the generated system control signal to the light source control section 34 and the image processing section 42.

The control section 44 includes a comparison determination section 44a. The comparison determination section 44a determines whether or not the size of a bleeding region is equal to or more than a predetermined value THA1 at the time of the white light mode, and whether or not the size of the bleeding region from the bleeding point is equal to or less than a predetermined value THA2 at the time of the deep blood vessel mode. The value THA2 is smaller than the value THA1.

More specifically, the comparison determination section 44a compares pixel values of respective pixels of red color in the endoscope image due to bleeding with a predetermined value THR1, calculates the size of a bleeding region from the number of pixels having the predeteimined value THR1 or more, and determines whether or not the size of the bleeding region is equal to or more than the predetermined value THA1 at the time of the white light mode. Further, the comparison determination section 44a compares the pixel values of the respective pixels of green color in the endoscope image due to bleeding with a predetermined value THR2, calculates a size of a region including the bleeding point in which a blood concentration is high from the number of pixels having the predetermined value THR2 or less, and determines whether or not the size of the region is equal to or less than the predetermined value THA2 at the time of the deep blood vessel mode. The region including the bleeding point is a region where only blood that is not diluted with water or the like exists, and has a high concentration of blood.

The control section 44 switches the observation image generation mode from a present observation image generation mode to the other observation image generation mode based on a determination result of the comparison determination section 44a. More specifically, the control section 44 switches the observation image generation mode to the deep blood vessel mode when the size of the bleeding region becomes equal to or more than the predetermined value THA1 at the time of the white light mode, and switches the observation image generation mode to the white light mode when the size of the region having a high concentration of blood becomes equal to or less than the predetermined value THA2 at the time of the deep blood vessel mode.

The display apparatus 5 includes an LCD (liquid crystal display), for example, and is configured to be able to display an observation image or the like that is outputted from the processor 4.

### (Operation)

Next, an operation and the like of the living body observation system 1 of the present embodiment will be described.

First, a user such as a surgeon connects the respective sections of the living body observation system 1 to input a power supply, and thereafter performs an instruction to set the observation mode of the living body observation system 1 at the white light mode by operating the input I/F 43.

The control section 44 generates a system control signal causing the R light, the G light and the B light to be simultaneously emitted from the light source apparatus 3 and outputs the system control signal to the light source control section 34, when the control section 44 detects that the observation mode is set at the white light mode, based on the instruction from the input I/F 43. Further, when the control section 44 detects that the observation image generation mode is set at the white light mode based on the instruction from the input I/F 43, the control section 44 generates a system control signal for causing an operation corresponding to the white light mode to be performed and outputs the system control signal to the resolution adjustment section 42B and the observation image generation section 42C.

The light source control section 34 performs control for bringing the red light source 31A, the green light source 31B and the blue light source 31C into a lighting state, and performs control for bringing the infrared light source 31D into an extinguishing state, based on the system control signal which is outputted from the control section 44.

Subsequently, the operation as described above is performed in the light source control section 34, whereby a WL light that is the white light including the R light, the G light and the B light is irradiated to the subject as the illuminating light, and a WLR light that is a reflected light emitted from the subject in response to irradiation of the WL light enters from the objective lens 17 as return light. Further, the WLR light which enters from the objective lens 17 is emitted to the camera unit 22 via the relay lens 18 and the eyepiece lens 19.

The dichroic mirror 23 transmits the WLR light emitted through the eyepiece lens 19 to the image pickup device 25A side.

The image pickup device 25A generates an image pickup signal by picking up an image of the WLR light which is transmitted through the dichroic mirror 23, and outputs the generated image pickup signal to the signal processing circuit 26.

The signal processing circuit 26 applies predetermined signal processes such as a correlated double sampling process and an A/D conversion process to the image pickup signal which is outputted from the image pickup device 25A, and thereby generates the image signal CS including the R image, the G image and the B image to output the generated image signal CS to the processor 4.

The color separation processing section 42A performs a color separation process for separating the image signal CS which is outputted from the endoscope 2 into the R image, the G image and the B image. Further, the color separation processing section 42A outputs the image signal RS corresponding to the R image obtained by the aforementioned color separation process, and the image signal BS corresponding to the B image which is obtained by the aforementioned color separation process to the resolution adjustment section 42B. Further, the color separation processing section 42A outputs the image signal GS corresponding to the G image which is obtained by the aforementioned color separation process to the observation image generation section 42C.

The resolution adjustment section 42B outputs the image signals RS and BS that are outputted from the color separation processing section 42A directly to the observation image generation section 42C based on the system control signal which is outputted from the control section 44.

The observation image generation section 42C generates an observation image by assigning the R image shown by the image signal RS which is outputted from the resolution adjustment section 42B to the R channel of the display apparatus 5, assigning the G image shown by the image signal GS which is outputted from the color separation processing section 42A to the G channel of the display apparatus 5, and assigning the B image shown by the image signal BS which is outputted from the resolution adjustment section 42B to the B channel of the display apparatus 5, and outputs the generated observation image to the display apparatus 5 based on the system control signal which is outputted from the control section 44. According to the operation of the observation image generation section 42C, the observation image including a color tone substantially similar to the case where the subject such as a biological tissue is seen with the naked eye is displayed on the display apparatus 5, for example.

The user can perform an instruction to set the observation image generation mode of the living body observation system 1 at the deep blood vessel mode by inserting the insertion portion 6 into the subject while confirming the observation image displayed on the display apparatus 5, and operating the input I/F 43 in the state in which the distal end portion of the insertion portion 6 is disposed in a vicinity of a desired observation site in the subject.

When the control section 44 detects that the observation image generation mode is set at the deep blood vessel mode based on the instruction from the input I/F 43, the control section 44 generates a system control signal for emitting the R light, the B light and the IR light simultaneously from the light source apparatus 3 and outputs the system control signal to the light source control section 34. Further, when the control section 44 detects that the observation image generation mode is set at the deep blood vessel mode based on the instruction from the input I/F 43, the control section 44 generates a system control signal for causing an operation corresponding to the deep blood vessel mode to be performed and outputs the system control signal to the resolution adjustment section 42B and the observation image generation section 42C.

The light source control section 34 performs control for bringing the red light source 31A, the blue light source 31C and the infrared light source 31D into a lighting state, and performs control for bringing the green light source 31B into an extinguishing state, based on the system control signal which is outputted from the control section 44.

Subsequently, the operation as described above is performed in the light source control section 34, whereby SL light which is the illuminating light including the R light, the B light and the IR light is irradiated to the subject, and SLR light which is the reflected light emitted from the subject in response to irradiation of the SL light enters from the objective lens 17 as the return light. Further, the SLR light which enters from the objective lens 17 is emitted to the camera unit 22 through the relay lens 18 and the eyepiece lens 19.

The dichroic mirror 23 transmits the R light and the B light included in the SLR light which is emitted through the eyepiece lens 19 to the image pickup device 25A side, and reflects the IR light included in the SLR light to the image pickup device 25B side.

The image pickup device 25A generates an image pickup signal by picking up an image of the R light and the B light which are transmitted through the dichroic mirror 23, and outputs the generated image pickup signal to the signal processing circuit 26.

The image pickup device 25B generates an image pickup signal by picking up an image of the IR light reflected by the dichroic mirror 23, and outputs the generated image pickup signal to the signal processing circuit 26.

The signal processing circuit 26 generates the image signal CS including the R image and the B image by applying the predetermined signal processes such as the correlated double sampling process and the A/D conversion process to the image pickup signal which is outputted from the image pickup device 25A, and outputs the generated image signal CS to the processor 4. Further, the signal processing circuit 26 generates the image signal IRS corresponding to the IR image by applying the predetermined signal processes such as the correlated double sampling process and the A/D conversion process to the image pickup signal which is outputted from the image pickup device 25B, and outputs the generated image signal IRS to the processor 4.

The color separation processing section 42A performs a color separation process for separating the image signal CS which is outputted from the endoscope 2 into the R image and the B image. Further, the color separation processing section 42A outputs the image signal RS corresponding to the R image which is obtained by the aforementioned color separation process, and the image signal BS corresponding to the B image which is obtained by the aforementioned color separation process to the resolution adjustment section 42B.

The resolution adjustment section 42B outputs the image signal IRS which is outputted from the endoscope 2 directly to the observation image generation section 42C based on the system control signal which is outputted from the control section 44. Further, the resolution adjustment section 42B performs a pixel interpolation process for increasing the resolution RA of the R image shown by the image signal RS which is outputted from the color separation processing section 42A to the resolution RB based on the system control signal which is outputted from the control section 44, generates the image signal ARS corresponding to the R image to which the pixel interpolation process is applied, and outputs the generated image signal ARS to the observation image generation section 42C. Further, the resolution adjustment section 42B performs a pixel interpolation process for increasing the resolution RA of the B image which is shown by the image signal BS which is outputted from the color separation processing section 42A to the resolution RB based on the system control signal which is outputted from the control section 44, generates the image signal ABS corresponding to the B image to which the pixel interpolation process is applied, and outputs the generated image signal ABS to the observation image generation section 42C.

The observation image generation section 42C generates the observation image by assigning the IR image shown by the image signal IRS which is outputted from the resolution adjustment section 42B to the R channel of the display apparatus 5, assigning the R image shown by the image signal RS which is outputted from the resolution adjustment section 42B to the G channel of the display apparatus 5, and assigning the B image shown by the image signal BS which is outputted from the resolution adjustment section 42B to the B channel of the display apparatus 5, based on the system control signal which is outputted from the control section 44, and outputs the generated observation image to the display apparatus 5. According to the operation of the observation image generation section 42C like this, for example, the observation image in which a blood vessel with a large diameter existing in a deep part of the biological tissue is emphasized in accordance with a contrast ratio of the R image and the IR image is displayed on the display apparatus 5.

According to the above living body observation system 1, when the user sets a desired observation image generation mode, the observation image generated in the set observation image generation mode is displayed on the display apparatus 5, whereas when the user performs treatment to the biological tissue, switch of the observation image generation mode between the white light mode and the deep blood vessel mode is performed automatically.

For example, the user inputs information indicating that treatment is going to be performed, that is, treatment start information to the control section 44 by performing a predetermined operation to the input I/F 43.

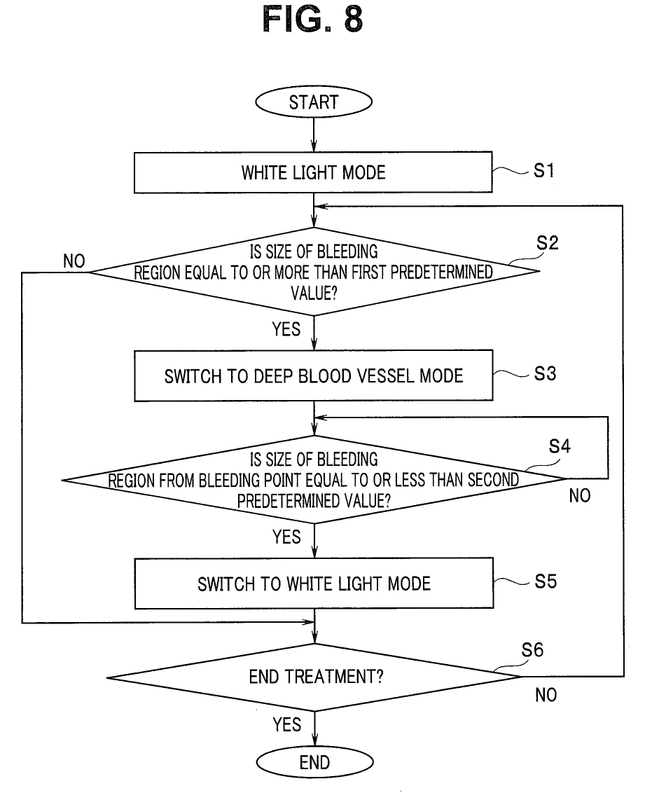

FIG. 8 is a flowchart illustrating an example of a flow of a switching process of the observation mode by the control section 44.

When the treatment start information is inputted, the control section 44 drives the image processing section 42 and the light source apparatus 3 in the white light mode (step (abbreviated as S hereinafter) 1).

The comparison determination section 44a determines whether or not the size of the bleeding region is equal to or more than the first value THA1 based on the observation image which is outputted by the observation image generation section 42C (S2).

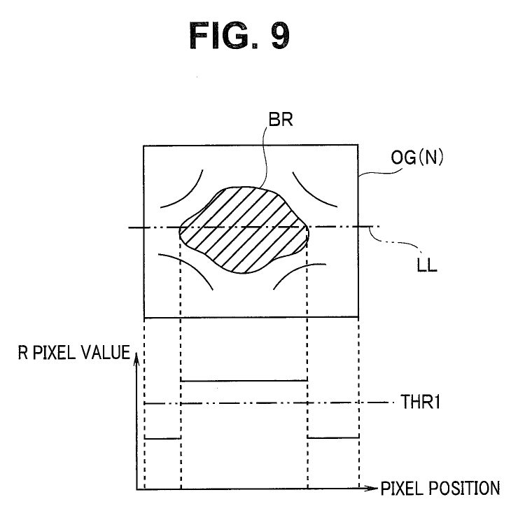

FIG. 9 is a diagram for explaining a bleeding region in an observation image OG (N) in the white light observation mode. FIG. 9 illustrates the observation image OG (N), and a graph of pixel values of respective pixels in one horizontal line LL in the R image of the observation image OG (N). In the observation image OG (N) in FIG. 9, a bleeding region BR is shown by oblique lines.

The observation image OG (N) which is the endoscope image is formed of three images of the R image, the G image and the B image. The comparison determination section 44a compares the pixel values of the respective pixels in the R image in the observation image OG (N) and the value THR1, extracts the pixels each having the pixel value equal to or more than the value THR1, and calculates the size of the bleeding region BR in the observation image OG (N) from a number of the extracted pixels. The comparison determination section 44a determines whether or not the calculated size S of the bleeding region BR is equal to or more than the value THA1.

When the horizontal line LL in the R image in the observation image OG (N) includes the pixels of the bleeding region BR as illustrated in FIG. 9, the pixel values of the respective pixels in the bleeding region BR are larger than the pixel values of the pixels in regions other than the pixels in the bleeding region BR. Therefore, the comparison determination section 44a can obtain the size S of the bleeding region BR by comparing the pixel values of the respective pixels on all horizontal lines in the R image and the value THR1.

As above, the image processing section 42 generates a color image in which the red signal, the green signal and the blue signal included in the image pickup signal generated in the image pickup device 25A are respectively assigned to the R channel corresponding to the red of the display apparatus 5 which displays a white light observation image, the G channel corresponding to the green of the display apparatus 5, and the B channel corresponding to the blue of the display apparatus 5 in the white light mode, and the control section 44 calculates the size of the region of blood based on the pixel values of the image pickup signal assigned to the R channel corresponding to the red of the display apparatus 5 in the white light mode, and determines whether or not the size of the region of blood is equal to or more than the value THA1.

Note that a halation region may be included in the endoscope image, so that pixels in the halation region may be excluded from the pixels of the bleeding region BR. For example, even when the pixel value of the R image is equal to or more than the value THR1, if the respective pixel values of the pixel in the G image and the pixel in the B image at a position corresponding to that pixel are each equal to or more than a predetermined value, that pixel in the R image is determined as the pixel in the halation region, and a process of not including that pixel in the pixels for calculating the size of the bleeding region BR may be performed. Alternatively, even when the pixel value of the pixel in the R image is equal to or more than the value THR1, that pixel in the R image is determined as the pixel in the halation region, based on a ratio of respective pixel values of the pixel in the G image and the pixel in the B image in a position corresponding to that pixel, to that pixel value, and a process of not including that pixel in the R image in the pixels for calculating the size of the bleeding region BR may be performed.

When the size S of the bleeding region BR is equal to or more than the predetermined value THA1 (S2: YES), the control section 44 perforins switch to the deep blood vessel mode (S3).

At this time, the control section 44 outputs the system control signal that switches the observation image generation mode to the deep blood vessel mode to the light source apparatus 3 and the image processing section 42, and thereby switch to the deep blood vessel mode is performed.

More specifically, the control section 44 performs control of switching the illumination mode to the illumination mode for the deep blood vessel mode from the illumination mode for the white light mode to the light source apparatus 3 when the image processing section 42 is switched to the deep blood vessel mode from the white light mode by the control section 44.

As above, the control section 44 calculates the size of the region of blood based on the number of pixels of the region of blood in the color image generated by the image processing section 42 which is the color image generation section, and when the calculated size of the region of blood becomes equal to or more than the value THA1, the control section 44 performs control of switching the observation image generation mode in the image processing section 42 to the deep blood vessel mode which is the second observation image generation mode from the white light mode which is the first observation image generation mode.

Consequently, when bleeding occurs during treatment, the observation image generation mode is automatically switched to the deep blood vessel mode, so that the surgeon can quickly perform hemostasis treatment without performing a switching operation of the observation image generation mode.

Further, when the surgeon finished hemostasis treatment, the surgeon has to return the observation image generation mode to the white light mode to continue treatment which is performed before the hemostasis treatment. When hemostasis treatment frequently takes place, the surgeon has to repeat a switching operation of the observation image generation mode to the white light mode.

Therefore, in this case, when hemostasis is finished, a process of automatically switching the observation image generation mode to the white light mode from the deep blood vessel mode is performed.

Therefore, after S3, the comparison determination section 44a determines whether or not the size of the bleeding region from the bleeding point is equal to or less than the predetermined value THA2 based on the observation image outputted by the observation image generation section 42C (S4).

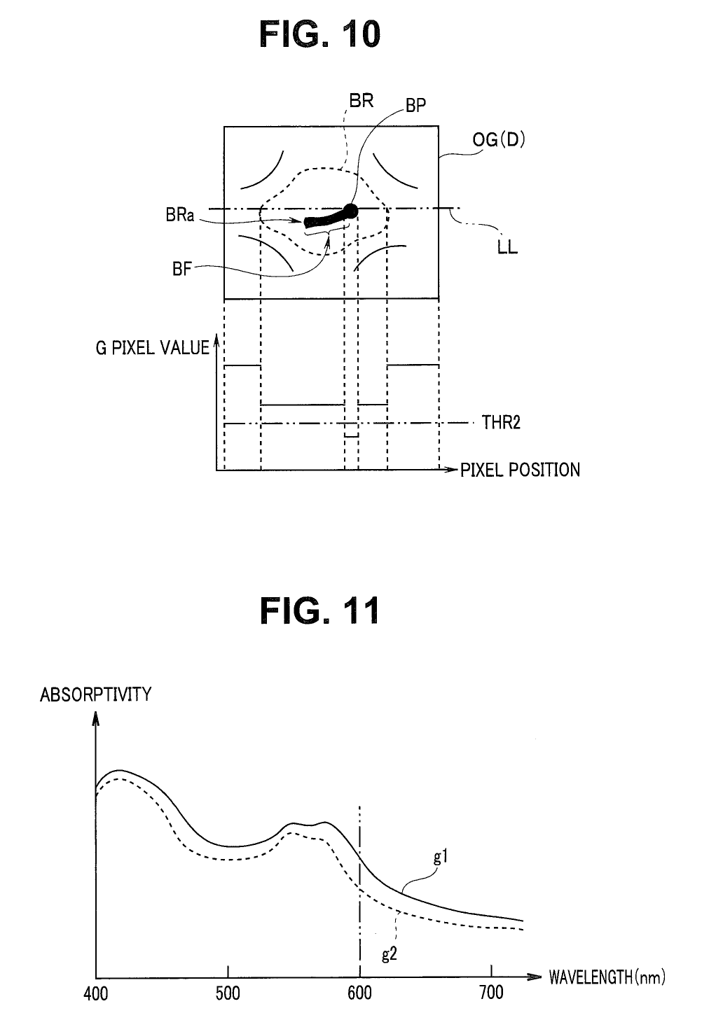

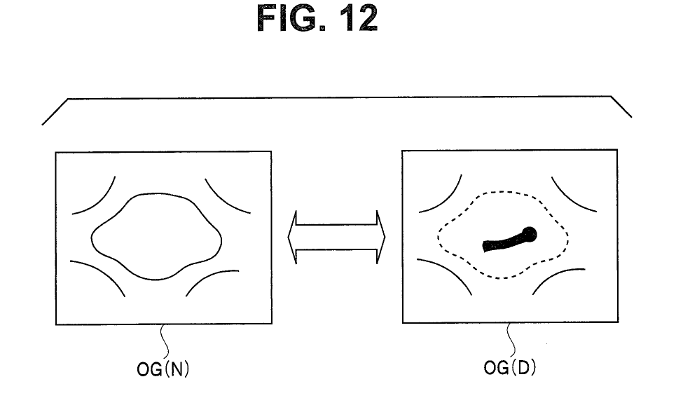

FIG. 10 is a diagram for explaining a bleeding region and a bleeding point in an observation image OG(D) in the deep blood vessel mode. FIG. 11 is a schematic graph illustrating light absorption characteristics of blood, to light wavelengths.

FIG. 10 illustrates the observation image OG (D), and a graph of pixel values of respective pixels in one horizontal line LL in the G image of the observation image OG(D). In the observation image OG(D) in FIG. 10, the bleeding region BR shown by dotted lines exists, and in the bleeding region BR, a bleeding point BP, and a flow BF of blood in which a concentration of the blood which flows out from the bleeding point BP is not reduced exist.

A vertical axis in FIG. 11 represents a molar absorption coefficient (cm-1/M), and a horizontal axis represents a wavelength. FIG. 11 illustrates a graph gl (shown by the solid line) illustrating a light absorption characteristic of only blood, and a graph g2 (shown by the dotted line) illustrating the light absorption characteristic of the blood diluted with water.

In general, venous blood includes an oxygenated hemoglobin (HbO2) and a reduced hemoglobin (Hb) (both are collectively referred to simply as hemoglobin hereinafter) at a ratio of approximately 60:40. Light is absorbed by hemoglobin, but an absorption coefficient differs for each wavelength of light. FIG. 11 illustrates a light absorption characteristic of the venous blood for each of wavelengths from 400 nm to approximately 700 nm. An absorptivity to light in a vicinity of a wavelength of 600 nm differs between the case of only blood (gl) and the case of blood diluted with water (g2). As illustrated in FIG. 11, the absorptivity of pure blood to the light in the vicinity of the wavelength of 600 nm is higher than an absorptivity of the blood diluted with water to the light in the vicinity of the wavelength of 600 nm.

The observation image OG (D) which is the endoscope image is a color image, and at the time of the deep blood vessel mode, the respective image signals are assigned to the respective channels of the display apparatus 5 as described above, and the R image shown by the image signal ARS which is outputted from the resolution adjustment section 42B is assigned to the G channel corresponding to the green of the display apparatus 5.

The comparison determination section 44a compares the pixel values of the respective pixels in the G image of the observation image OG (D) with the value THR2, extracts the pixels equivalent to or less than the value THR2, and calculates the size of the region where the concentration of blood is high (also referred to as a high concentration blood region hereinafter) BRa in the observation image OG from the number of extracted pixels equal to or less than the value THR2. The high concentration blood region BRa is the region of the bleeding point BP and the blood flow BF in FIG. 10. The comparison detenuination section 44a determines whether or not the calculated size Sa of the high concentration blood region BRa is equal to or less than the predetermined value THA2.

When the single horizontal line LL in the G image of the observation image OG includes the pixels of the high concentration blood region BRa as illustrated in FIG. 10, the pixel values of the pixels of the high concentration blood region BRa are smaller than pixel values of the pixels in regions other than the high concentration blood region BRa. This is because the absorptivity of only blood to the light in the vicinity of 600 nm is higher than the absorptivity of the blood diluted with water to the light in the vicinity of 600 nm, so that the green becomes weaker in the high concentration blood region BRa as illustrated in FIG. 11.

As illustrated in FIG. 10, the bleeding region BR displayed in red in the white light mode is transparent and becomes hard to see in the deep blood vessel mode, but the bleeding point BP and the blood flow portion BF that flows from the bleeding point BP is a high concentration blood region, where the green is weak, and is displayed in orange on the display apparatus 5. Note that in FIG. 10, the bleeding point BP and the blood flow portion BF are shown in black.

By comparing the pixel values of the respective pixels on all the horizontal lines in the G image with the value THR2, the size Sa of the high concentration blood region BRa is obtained as described above.

Subsequently, it is determined whether or not the obtained size Sa of the high concentration blood region BRa is equal to or less than the predetermined value THA2 (S4).

When the size Sa of the high concentration blood region BRa is not equal to or less than the predetermined value THA2 (S4: NO), hemostasis is not sufficient, so that the determination process in S4 is continued.

When the size Sa of the high concentration blood region BRa is equal to or less than the predetermined value THA2 (S4: YES), it is conceivable that hemostasis is completed, so that the control section 44 switches the observation image generation mode to the white light mode (S5).

More specifically, the control section 44 performs switch to the white color mode by outputting the system control signal for switching the observation image generation mode to the white light mode to the light source apparatus 3 and the image processing section 42.

As above, the image processing section 42 generates a color image in which the image pickup signal corresponding to the light with the center wavelength of 600 nm, the image pickup signal corresponding to the light with the center wavelength of 800 nm and the image pickup signal corresponding to the light with the center wavelength of 460 nm which are included in the image pickup signals generated in the image pickup devices 25A and 25B are respectively assigned to the G channel corresponding to the green of the display apparatus 5 displaying the deep blood vessel observation image, the R channel corresponding to the red of the display apparatus 5 and the B channel corresponding to the blue of the display apparatus 5, and the control section 44 calculates the size of the bleeding region from the bleeding point based on the pixel value of the image pickup signal assigned to the G channel corresponding to the green of the display apparatus 5 in the deep blood vessel mode, and when the size of the bleeding region from the bleeding point becomes equal to or less than the value THA2, the control section 44 performs control to switch the observation image generation mode in the image processing section 42 to the white light mode from the deep blood vessel mode.

Thereafter, it is determined whether or not there is an input for ending the treatment to the input I/F 43 (S6), and in the case of ending the treatment (S6: YES), the process is ended.

When the treatment is not ended (S6: NO), the process returns to S2.

Further, when the size of the bleeding region is not equal to or more than the predetermined value THR1 in S2, the process goes to S6.

FIG. 12 is a diagram for explaining a change of the observation image by switch of the observation mode. When there is no bleeding during treatment, the observation image OG (N) in the white light observation mode is displayed on the display apparatus 5. However, when bleeding occurs, and the size S of the bleeding region BR in the observation image OG (N) becomes equal to or more than the predetermined size, the observation image displayed on the display apparatus 5 is automatically switched to the observation image OG (D) in the deep blood vessel observation mode from the observation image OG (N).

In the observation image OG (D), the bleeding point BP can be visually recognized, so that the surgeon can immediately perform hemostasis treatment.

When the hemostasis treatment is perfoiiiied, and the size Sa of the high concentration blood region BRa becomes equal to or less than the predeteitnined size, the observation image which is displayed on the display apparatus 5 is automatically switched to the observation image OG (N) in the white light observation mode from the observation image OG (D).

Consequently, when hemostasis is ended, it is possible to continue treatment such as mucosal dissection immediately. As illustrated in FIG. 12, the observation image which is displayed on the display apparatus 5 is automatically switched between the observation image OG (N) in the white light observation mode and the observation image OG (D) in the deep blood vessel observation mode.

As above, according to the aforementioned embodiment, the living body observation system can be provided, which automatically performs switch to the observation mode in which visual recognition of the bleeding point is possible, without requiring a switching operation of the observation mode.

Note that in the aforementioned embodiment, the respective observation images in the white light observation mode and the deep blood vessel observation mode are generated from reflected lights from the object by irradiating a plurality of illuminating lights corresponding to the respective modes, but may be generated by image processing of so-called spectral estimation.

Furthermore, in the aforementioned light source apparatus 3, the LEDs or the like corresponding to the respective wavelength bands are continuously lighting, but illuminating lights of three colors corresponding to the observation mode may be sequentially irradiated by a frame-sequential method by using a white light source and a rotary filter.

Further, in the aforementioned embodiment, the endoscope is a rigid endoscope, but may be a flexible endoscope.

The present invention is not limited to the aforementioned embodiment, and various modifications, alterations and the like can be made within the range without changing the gist of the present invention.

What is claimed is:

1. A living body observation system comprising:
a light source section configured to be able to switch between a first illumination mode that emits light which is illuminating light for irradiating a subject to generate a first observation image, as the illuminating light, and a second illuminating mode that emits a light in a first wavelength band that is a red band in a visible region and is a narrow band between a wavelength band including a maximum value and a wavelength band including a minimum value in a hemoglobin light absorption characteristic of a biological tissue of the subject, a light in a second wavelength band that is a wavelength band having a longer wavelength than the first wavelength band and is a narrow band in which an absorption coefficient in the hemoglobin light absorption characteristic is lower than the light in the first wavelength band and a scattering characteristic of the biological tissue is suppressed, and a light in a third wavelength band of a shorter wavelength than the red band in the visible region, as the illuminating light;
an image pickup section including a plurality of pixels that receive light from the subject and generate an image pickup signal;
a color image generation section configured to include a first observation image generation mode that generates the first observation image of the subject from the image pickup signal, and a second observation image generation mode that generates a second observation image that is a deep blood vessel observation image that is for observing a deep blood vessel of the subject and is obtained when the light in the first wavelength band, the light in the second wavelength band, and the light in the third wavelength band are irradiated, as observation image generation modes, and generate color images of the subject in the first observation image generation mode and the second observation image generation mode respectively; and
a control section configured to calculate a size of a bleeding region from a bleeding point based on a pixel value of an image pickup signal corresponding to the light in the first wavelength band included in the image pickup signal generated in the image pickup section in the second observation image generation mode, and perform control to switch the observation image generation mode in the color image generation section to the first observation image generation mode from the second observation image generation mode when the size of the bleeding region from the bleeding point becomes equal to or less than a first value, and switch the observation image generation mode in the color image generation section to the second observation image generation mode from the first observation image generation mode when the size of the bleeding region in the color image that is generated by the color image generation section becomes equal to or more than a second value.

2. The living body observation system according to claim 1,
wherein the control section calculates a number of pixels in the bleeding region in the color image, determines whether or not the size of the bleeding region is equal to or more than the second value based on the calculated number of pixels, and performs control to switch the observation image generation mode in the color image generation section to the second observation image generation mode from the first observation image generation mode when the size of the bleeding region is equal to or more than the second value as a result of determination.

3. The living body observation system according to claim 1,
wherein the first observation image generation mode is a white light observation image generation mode that generates a white light observation image that is obtained when the subject is irradiated with white light as the illumination light.

4. The living body observation system according to claim 3,
wherein the color image generation section generates a color image in which a red signal, a green signal and a blue signal that are included in the image pickup signal generated in the image pickup section are respectively assigned to a channel corresponding to red color of a display apparatus that displays the first observation image, a channel corresponding to green color of the display apparatus and a channel corresponding to blue color of the display apparatus, in the white light observation image generation mode, and
the control section determines whether or not the size of the bleeding region is equal to or more than the second value based on a pixel value of an image pickup signal assigned to the channel corresponding to red color of the display apparatus, in the white light observation image generation mode.

5. The living body observation system according to claim 1,
wherein the first value is smaller than the second value.
